(19) United States
(12) Patent Application Publication
(10) Pub. No.: US 2020/0016591 A1
(43) Pub. Date: Jan. 16, 2020

(54) A Molecule Printing Device for the Analysis of the Secretome of Single Cells

(71) Applicant: University of Twente, Enschede (NL)

(72) Inventors: Richard Schasfoort, Haaksbergen (NL); Arjan Tibbe, Deventer (NL); Joska Johannes Broekmaat, Enschede (NL); Fikri Abali, Enschede (NL); Leon WMM Terstappen, Amsterdam (NL)

(73) Assignee: University of Twente, Enschede (NL)

(21) Appl. No.: 16/465,686

(22) PCT Filed: Dec. 14, 2017
(86) PCT No.: PCT/EP2017/082936

§ 371 Date: May 31, 2019

Related U.S. Application Data
(60) Provisional application No. 62/434,492, filed on Dec. 15, 2016.
(60) Provisional application No. 62/569,646, filed on Oct. 9, 2017.

(51) Int. Cl. B01L 3/00; G01N 33/543

(52) U.S. Cl. CPC B01L 3/50857; B01L 3/50255; B01L 2300/0829; B01L 3/50273; B01L 3/502715; G01N 33/54373

(57) ABSTRACT

The present invention discloses a device and methods for the analysis of secreted molecules from a single cell. As described herein, the invention incorporates individual microwells with a bottom surface capable of capturing a cell and allows the release of secreted molecules to be printed onto a capture surface. The device provides accurate identification of the cell source of the printed molecules by mapping the printed molecules to the cell source. The invention further employs spectrometry, immunoassay or label free Surface Plasmon Resonance imaging for detection of the secreted molecules in combination with a microwell array, where single cells are seeded in individual microwells and the secreted molecules are captured by the capture surface in an array print while the cell remains in the microwell for additional interrogation. The present invention has applications in medical research and diagnosis where individual target cells in a fluid sample are interrogated for the secreted products.

### CROSS REFERENCE TO RELATED APPLICATIONS

This application is the US national application of PCT/EP2017/082936, filed on 14 Dec. 2017, which claims priority to U.S. Provisional Applications 62/434,492, filed on 15 Dec. 2016, now expired, and U.S. 62/569,646, filed on 9 Oct. 2017, now expired, the disclosures of which are herein incorporated by reference in its entirety.

### BACKGROUND

### Field of Invention

The present invention relates to a device for the analysis of secreted products by single cells. More specifically, the present invention relates to a method and device coupling a chip containing microwells with a surface that captures the released products. Cells encompass both eukaryotic (presence of a nucleus) and prokaryotic cells (without a nucleus). Examples are hematopoietic cells, epithelial cells, mesodermal cells, cancer cells, organoids, bacteria, algae, and plant cells. The individual microwells in the chip contain one single cell and the secreted product of each cell is printed onto a capture surface through the pore(s) in the bottom of the microwell. The secreted products on the capture surface can be detected by various means such as Surface Plasmon Resonance imaging, fluorescence microscopy, mass spectroscopy, ELISA, spectroscopy, or other methods to detect molecules on a surface. As a capture surface functionalized SPR sensors, functionalized glass surfaces, functionalized ceramic or metal surfaces, functionalized polyvinylidene difluoride (PVDF)-membrane surfaces or other functionalized and non-functionalized surfaces to capture molecules area, which are known in the field of the invention. Simultaneous detection of various molecules secreted from a single cell at different time points is a variant of the application that can be used to characterize the single cell.

### Description of Related Art

Single cell technologies are of extreme importance when characteristics of individual cells need to be assessed or differences between cells need to be elucidated. Applications range from cells that are extremely rare such as Circulating Tumor Cells (CTC) in blood or abundant such as hybridoma cells producing monoclonal antibodies. Technologies commonly used to identify and sort individual cells include fluorescence activated cell sorting (FACS), laser-capture microdissection, cell picking using micropipettes limited dilution sedimentation in wells, magnetic rafts and a variety of microfluidic chips with different structures and different underlying cell isolation principles. Although these technologies enable the isolation of individual cells they do not enable the measurement of the products secreted by the cells.

The vast majority of molecules in bodily fluids such as lipids and proteins are produced by individual cells. The most abundant protein in human plasma is albumin produced by cells in the liver, other proteins are for example antibodies produced by plasma cells in bone marrow and lymph nodes, hormones secreted by cells in the endocrine glands and cytokines small proteins (˜5-20 kDa) important for cell signaling and produced by a broad range of cells such as lymphocytes, macrophages and stromal cells. To actual study the secretome of individual cells and study which factors influence the secretion, technology is needed that permits the assessment of the secreted products on an individual cell basis and if possible over time. A large variety of questions can be answered with the availability of such tools. Examples of medical related questions to be answered are, the production of hormones like insulin in beta cells present in pancreatic islets to ultimately cure/treat diabetes. The secretome of individual cancer cells to identify those molecules responsible for the spread of the disease (metastatic process) thereby increasing our chance to find effective therapies. Other examples relate to the production of molecules by bioengineered cells like the production of therapeutic antibodies by cells followed by the isolation of cells that produce the therapeutic antibody most efficiently for clonal expansion and the production of coloring agents and lipids by algae for selecting the algae that produce the molecules of interest most efficiently. In addition, factors influencing the production can be optimized by measuring the production rate after modification of the growth conditions. The choice of the technology used to analyze the secretion products depends on the application.

The principle of the self-sorting microwell chip has been previously described (U.S. Pat. No. 9,638,636, Feb. 5, 2016, J. F. Swennenhuis et al., Lab Chip, 2015, 15, 3039-3046). In brief, the chip comprise microwells present in a supporting silicon substrate where each microwell is closed by thin silicon nitride membrane that contains precisely etched pores. The membranes are mechanically stable and can withstand high pressure at a thickness of only a few hundred nanometers.

Typically, the self-seeding microwell chip comprises of 6400 microwells in an effective area of 8×8 mm². Each microwell has a diameter of 70±2 μm, a depth of 360±10 μm with a well volume of 1.4 nL. The bottom of the microwell is a thin, optically transparent, silicon nitride (SiN) membrane with a thickness of 1 μm, having a single pore with a diameter of 5 μm in the bottom. The sample liquid is filtered through the pores with low flow resistance allowing for high flow rates. The cells or microorganisms are dragged by fluidic forces into the microwell. Once a cell has landed onto the pore of one of the microwells, the flow is significantly reduced forcing the other cells towards the adjacent wells. This results in forced single cell seeding in individual wells. After identification of the cells of interest they can be isolated from the microwell by punching the bottom out including the cell towards a reaction tube, well plate or other format optimal for the intended application.

### SUMMARY

The present invention resolves the limitations of the prior art by combing a method to separate and handle single cells in individual microwells, with the ability to print the secreted product from these cells onto a surface, while these are present and alive in the microwells. In addition, the location of the printed molecules can be related back to the microwell number and the cell in this particular microwell can be isolated. This enables the parallel monitoring of the secretion of thousands of individual cells with the ability to isolate the individual cells of interest for further interrogation or propagation.

The present disclosure teaches the printing of the microwell content onto a capture surface and illustrates the principles through measurement of its content by Surface Plasmon Resonance imaging (SPRi) and immunoassay (IA), followed by isolation of the cells of interest from the microwells. Accurate printing of the microwell content is critical. To be able to accurately measure the amount of secreted molecules it is required to establish a contact between the microwell bottoms and capture membrane that is equal for all microwells. Depending on the method of analysis of the printed molecules, the microwells with the cells are separated from the capturing surface or can be left in contact with the capturing surface during analysis. Selection criteria for the cells based on the printed molecules are the amount of produced molecules and the quality of the produced molecules. After completion of the printing process and analysis of the printed molecules, the well number belonging to the printed dot with the required properties is identified. The bottom and cell of the identified microwell is punched towards the reaction tube of choice or culture plate for further expansion of the selected cell. One embodiment of the invention is the use of microwells as a transport unit to enable the use of multiple capturing surfaces for the same cells. In this particular case, the microwell chip with the single cells in the individual microwells is pushed onto the first surface and after the amount of molecules is large enough the microwell chip is removed from surface one and placed onto capturing surface two. This can be continued multiple times with the advantage that the secretion of individual cells can be determined using different capture surface and analysis methods.

Another embodiment of the present invention is the use of the microwell chip as a printing unit for printing molecules. In this case, the microwells are filled with a solution containing molecules. These molecules can be printed on the surface by pushing the microwell onto the capturing surface. This is of interest for generating surfaces that need to be provided with molecules at specified locations in a confined area. Another possibility is to first capture the individual cells and lyse the cells such that the content of the single cell is printed on the capturing surface or multiple surfaces.

### BRIEF DESCRIPTION OF THE FIGURES

FIG. 1 Principle of the micro well device

Panel A displays the distribution of single cells in individual wells of a microwell chip (1). Cells in suspension follow the flow lines and as soon as a cell (2) has landed on the pore the flow through that particular pore stops and the other cells are diverted to the next available well. Panel B, present schematically the microwells through which a cell suspension (4) has passed and to which a capturing surface (3) is brought in contact with the bottom of the microwell chip (1). The insert shows an enlarged view of the bottom of the well. The viable cells inside the microwells secrete molecules (6), which are captured by molecules (5) attached to the capturing surface that can bind the secreted molecules. In panel C, the membrane is detached from the microwells and the composition of the printed spots (7) can be analyzed. Next, the location of the spots is correlated with the microwell number in which the cell resides that produced the content of the particular spots. Panel D present the principle of isolation. The cells in the microwells can now be punched with a needle (8) into a reaction/culture plate (9) by both the bottom of the microwell and the cell are now in the reaction/culture plate (9).

FIG. 2 Stitched fluorescent images of PE labeled antibodies on a PVDF membrane (A, B) and glass surface (C, D) after contact with the microwell chip containing a suspension of PE labeled antibodies. Images were taken using excitation and emission cubes for Phycoerythrin using a 480 nm LED as excitation source and a 10λ (NA0.45) objective. The imprint of the microwell chip containing 6400 wells on an area of 8×8 mm square is clearly visible. The squares in the images represent the borders of the stitched images. The inserts B and D show a larger magnification of the area in A and C, respectively.

FIG. 3 Microwell chip roadmap of the capture surface. Panel A stitched images taken with a fluorescent microscope of a PVDF surface after being in contact with the microwell chip. Images were taken using excitation and emission cubes for Allophycocyanin using a 630 nm LED as excitation source and a 10λ (NA0.45) objective. The insert in panel B shows a higher magnification of an area of the insert. Panel C shows an image taken by SPRi of the SPR Sensor surface with the microwells pressed in the evanescent field of a hydrogel coated (100 nm) SensEye sensor. The camera of the IBIS MX96 SPR imager used for this experiment is not large enough to cover the entire microwell chip (˜⅔ of the microwells covered). The insert in panel D inserts shows a higher magnification of an area of the insert.

FIG. 4 Stitched fluorescent images of the VU1D9 monoclonal antibody printed from the microwell chip containing a suspension of VU1D9 antibodies onto a PVDF membrane (A). The antibodies are visualized by staining the PVDF membrane with PE labeled anti-immunoglobulin (IgG) antibodies. The insert shows a higher magnification of one area (B). Image was taken using excitation and emission cubes for PE using a 480 nm LED as excitation source and a 10× (NA0.45) objective.

FIG. 5 Panel A shows an overlay of stitched fluorescent images of viable VU1D9 hybridoma cells (white) in the wells of the microwell chip and the VU1D9 antibodies onto the recombinant EpCAM coated PVDF membrane (gray). The antibodies are visualized by staining the PVDF membrane with PE labeled IgG antibodies. The insert in panel B shows a higher magnification of one area. Image was taken using excitation and emission cubes for PE using a 480 nm LED as excitation source and a 10× (NA0.45) objective.

FIG. 6 The principle of the microwell device using SPRi as a read out. The microwells (1) with single cells (2) are attached to a SPR sensor surface (10) and immersed in cultivation medium (4). The product of the cells (6) diffuse via the pore in the 1 μm bottom membrane to the sensor surface coated with a ligand (5) of the cell product (6) which is placed at a distance of −1 μm using edge spacers. The cell production can be measured by SPRi (11) real time as illustrated in this figure or after detaching the capture surface from the microwells.

FIG. 7 SPR image (referenced) of VU1D9 antibodies secreted in the microwells and captured to the sensor surface.

FIG. 8 A prototype of the fluidic device is shown in Panel A, the cup containing the microwell chip on the bottom and Panel B, the cap with which the cup can be sealed. Panel C, D and E show schematic representations of the mounting onto an SPRi sensor with Panel C, D (top view) and E (cross section).

FIG. 9 Schematic representation of the coupling device. Panels A, B and C show three different cross sections of the device. Panel A shows the coupling device (17) with the cell suspension (6), the microwell chip (12), O-rings at different positions (13, 14, 15) and the sealing tape (16). Panel B shows the attachment of the coupling device to the capture surface (10) using a head (18) on top of the cap (19) to create the force needed to press the microwell chip onto the SPR surface (10). Panel C shows a cross section of the coupling device.

FIG. 10 A coupling device to capture molecules secreted by single cells onto a PVDF membrane. A microwell chip (1) is mounted in a plastic holder (21). The PVDF membrane (3) is placed on a rubber slab (20) and on top of this slab the microwells are placed such that the PVDF membrane fits exactly on the microwell bottom. The stack is placed on a clamping unit (22). By closing the unit with the handle (23) the microwells are pushed down onto the PVDF membrane.

FIG. 11 Artist impression of three of the 6400 wells in the microwell chip (upper part) in which the secreted molecules from the single cells are diffused and captured on four different functionalized spots (A, B, C, D) on the capture surface (lower part). The A, B, C, D array is the capture array on the sensor surface and 4 secretion factors per cell can simultaneously be assessed. Each corner of the printed array is exposed to the hole in the microwell. If the first hole is exposed to A, B, C, D then the second hole will be in contact with B, A, D, C. etc. The array and capture area is not aligned in this image. Measurement of the molecules can be performed by SPRi real-time and by sensing techniques such as immune fluorescence, RAMAN spectroscopy and Mass spectrometry at different intervals during the production process.

### DETAILED DESCRIPTION OF INVENTION

This invention enables the monitoring of cellular secretion for thousands of cells in parallel and enables the selection and isolation of the cell of interest based on their secreted product. The invention results in a relatively simple protocol and workflow to monitor, track and quantify the secretion of molecules by single cells.

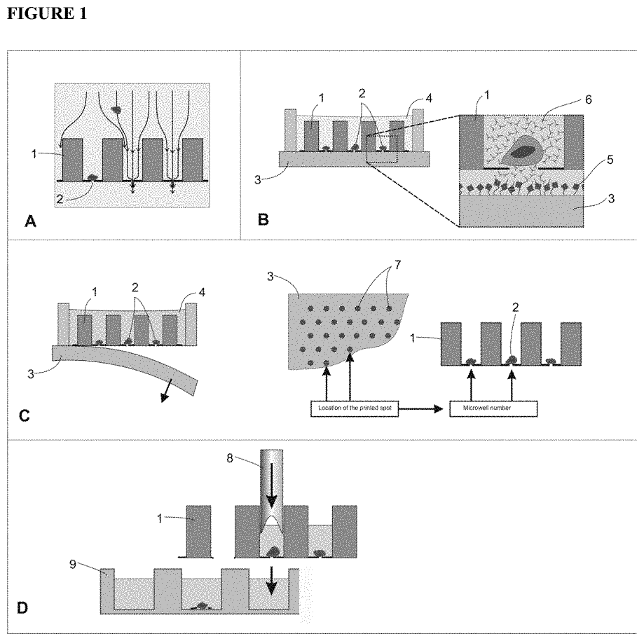

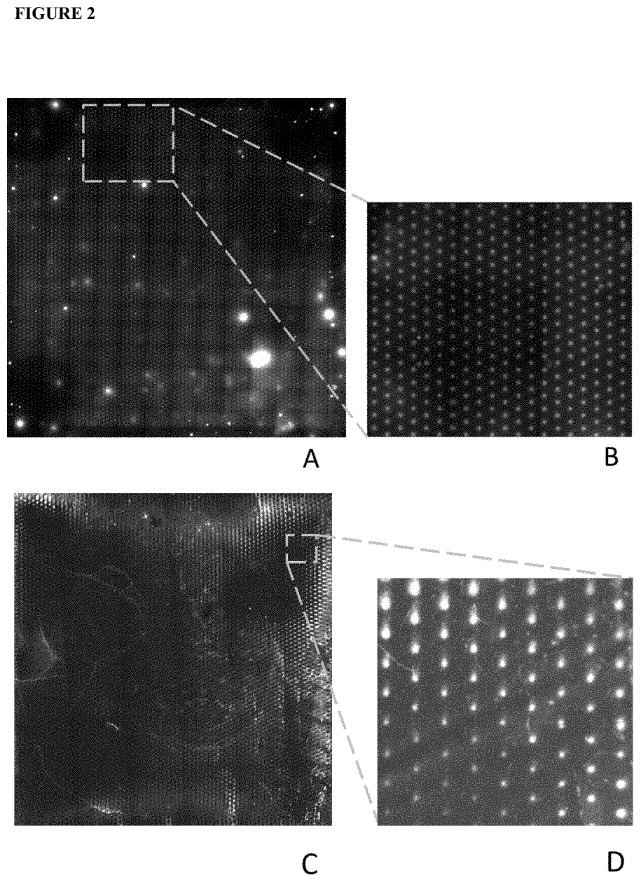

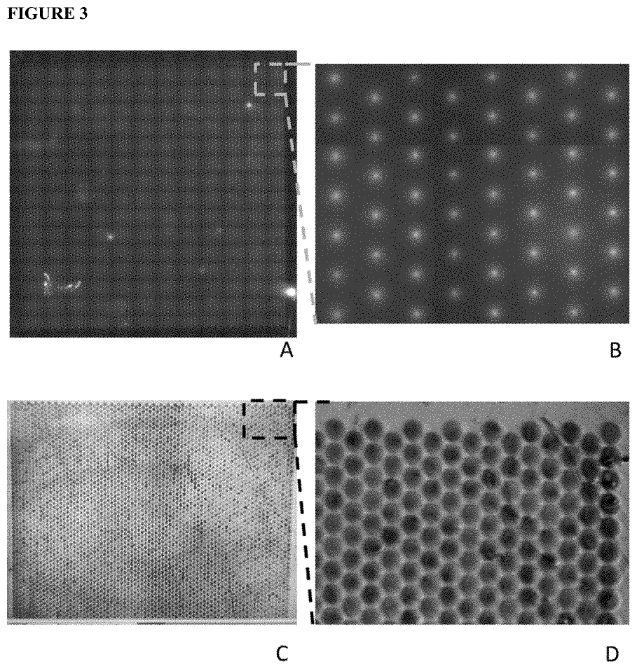

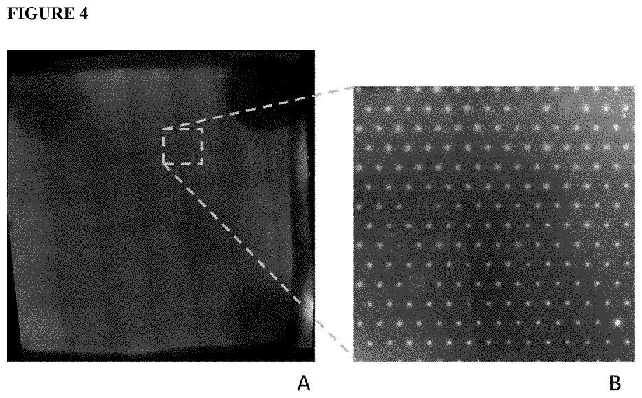

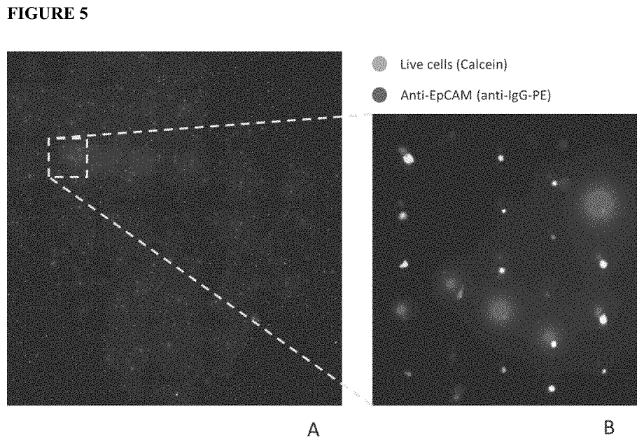

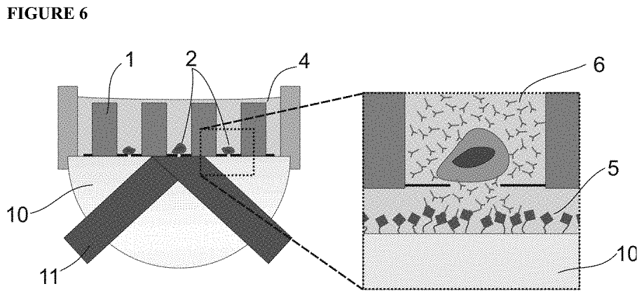

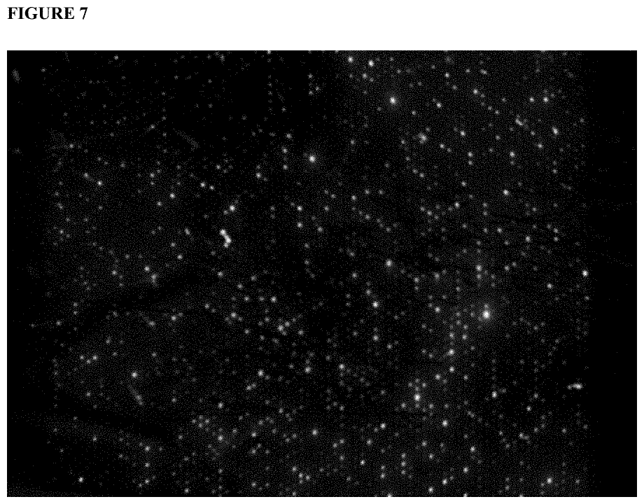

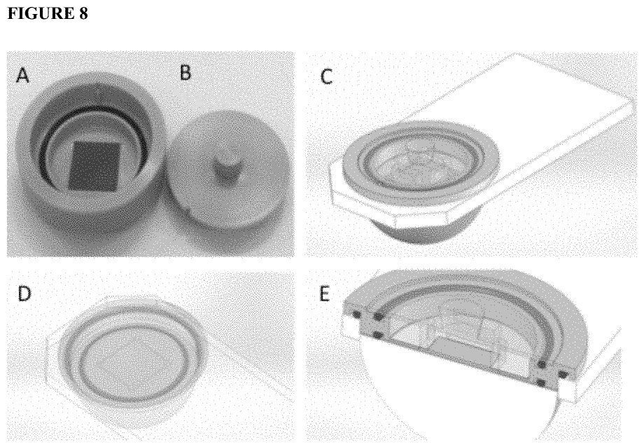

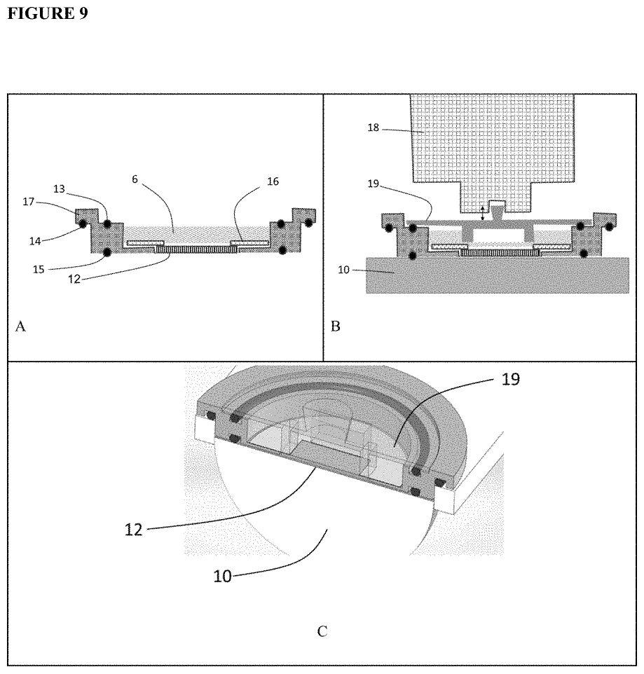

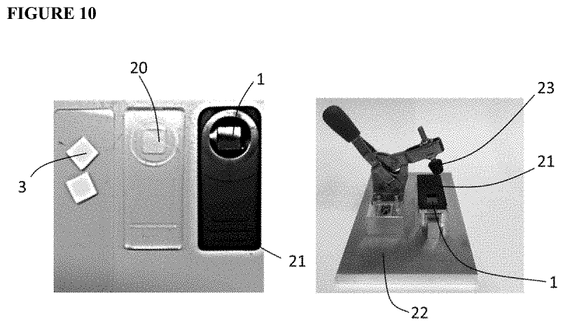

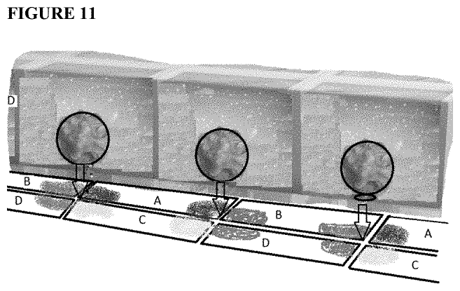

The principle of the invention is illustrated in FIG. 1. A cell suspension is transferred to the supply side of the microwell chip. To facilitate easy handling of the microwell chip the chip is mounted onto a plastic like material. By applying a small negative pressure at the bottom side of the microwells chip the cell suspension fluid enters the microwells and exits through the pore in the bottom of the microwells. The cells present in the cell suspension are dragged by fluidic forces into the microwells and pulled towards the pore(s) in the membrane at the bottom of the microwell. Once the cell lands onto the pore it occludes the pore and the flow through that pore stops. The remaining fluid and cells will be diverted to the next available microwell resulting in a fast distribution of single cells in individual microwells (U.S. Pat. No. 9,638,636, Feb. 5, 2016, J. F. Swennenhuis et al., Lab Chip, 2015, 15, 3039-3046). Panel A of FIG. 1 depicts a representation of 3 of the 6400 wells each with one cell blocking the hole in the bottom. The medium in which the cells are contained is chosen such that it propagates the secretion of molecules of the cells of interest and the chip is placed in an environment optimized to maintain cell viability. After completion of the cell seeding, the microwell chip with the pore is placed into contact with the capturing surface with the bottom of the microwells facing the capturing surface (Panel B). A thin fluid layer with a thickness of less than 1 micron enables the transport by diffusion of secreted molecules towards the capturing surface. In principle there is no flow from the microwells towards the capturing surface and the molecules secreted by the cells reach the surface by diffusion. In case of a flow the secreted molecules will be spread across a larger surface which will result in a loss of the relation printed spots and microwell number as well as reduced number of molecules per surface area which results in lower signal to noise ratio. However a flow rate that is so small that it allows binding of the molecules to the capture surface in the area of the pore without interfering with the neighboring spots is allowed. The insert in Panel B shows the production of antibodies by the cell and the diffusion and capture of these antibodies to the capture surface. The capture surface can now be detached from the bottom of the chip and the content analyzed, Panel C. This process can be repeated multiple times and over shorter or longer periods of time or onto different capturing surfaces. The coordinates of the microwells containing cells and the printed spots on the capture surface need to be correlated to determine which microwell belongs to the printed spots, Panel C. Methods to correlate the location of the printed spots with the well/cell number are disclosed below. After this relation has been determined the cells of particular interest based on the analysis of the produced molecules can now be isolated as depicted in Panel D. The latter method has been disclosed previously cells (U.S. Pat. No. 9,638,636, Feb. 5, 2016, J. F. Swennenhuis et al., Lab Chip, 2015, 15, 3039-3046).

Printing of the molecules that reside in the fluid present inside the microwell onto a captured surface is illustrated by applying a fluid containing Phycoerythrin (PE) conjugated antibodies onto the microwell chip. FIG. 2 shows an image of the printed spots onto a capture surface. The insert in FIG. 2B show a higher magnification of an area on the chip and illustrates the confinement of the PE antibodies to the areas corresponding to the holes in the bottom of the wells.

Although the print illustrated in FIG. 2 will allow one to match the position of each printed spot with the microwell number, this roadmap would need a marker in the fluid in which the cells reside which may interfere with the secreted products and available binding sites on the capturing surface. We overcame this issue by the introduction of a much simpler solution as is illustrated in FIG. 3. Panel A shows the stitched images taken from a PVDF membrane after it has been brought in contact with the microwell chip and a small pressure (0-20 N/10 mm² chip surface) is applied. The pressure of the micro well chip results in a stable indentation of the PVDF membrane material which results in a local fluorescence and photoluminescence change that is clearly visible using fluorescence microscopy. The indentation is already clearly visible on the PVDF membrane without using additional reagents such as nanoparticles and fluorescence dyes. Since all microwells show up in the indentation, the correlation between captured molecules and microwell number can be established and the cells of interest can be isolated. The insert in FIG. 3B shows a higher magnification of an area on the PVDF membrane nicely showing the position of the individual microwells.

Panel C shows an image from a SPRi sensor prism surface taken on a IBIS MX96 SPR-imager while the microwells are pressed in the evanescent field of a hydrogel coated (100 nm) SensEye sensor. To obtain the image of the membrane in the microwells, the membrane needs to be in the evanescent field of the light that scans the SPRi surface. The insert in FIG. 3D shows a higher magnification of an area and image clearly shows the position of the individual wells, which can be used to identify the microwells that printed the spots and contain the cells of interest. In this case the number of microwells visible is limited by the optical design and size of the CCD chip present in the SPR-imager.

To demonstrate that one can identify molecules derived from single wells onto a PVDF membrane a solution containing monoclonal antibody VU1DG was passed through a microwell chip and collected on an activated PVDF membrane that was in contact with the membrane side of the microwells. After removing the membrane from the microwell chip, the PVDF membrane was labeled with R-Phycoerythrin (PE) conjugated anti-immunoglobulin (IgG). After incubation and washing, the membrane was placed on a fluorescent microscope and images were taken of an area of 10×10 mm², to cover the entire microwell chip. FIG. 4A presents the stitched fluorescent images of IgG-PE labeled printed VU1D9 monoclonal antibody spots printed from the microwell chip. FIG. 4B displays an enlarged area of this image.

Next it was demonstrated that VU1D9 antibody produced by individual VU1D9 hybridoma cells can be detected on the PVDF membrane. A PVDF membrane was activated by coating with recombinant EpCAM. Hybridoma cells were first stained with the fluorescent dye Calcein (stains living cells) followed by passing the cell suspension through the microwell chip. The microwell chip with single hybridoma cells in individual wells was placed on an inverted fluorescence microscope and images were acquired of the entire microwell chip. Next the microwell chip was place on top of the recombinant EpCAM labeled PVDF membrane with the pores facing the PVDF membrane. A small pressure of around 0.1 N/mm2 was applied to enable proper contact between the microwell chip and the PVDF membrane to allow printing of the cell secreted EpCAM antibodies onto the membrane as well as to create an indentation of the microwells chip in the PVDF membrane. The stack of micowell chip (containing the cells) and membrane were placed and kept overnight in an incubator at 37° C. After incubation, the PVDF membrane was removed from the microwell chip and the PVDF membrane was incubated with PE labeled anti-immunoglobulin (IgG) antibodies. After washing the membrane with a solution of PBS and BSA, the PVDF membrane was placed on the inverted fluorescence microscope and fluorescence images were acquired to cover the surface of the PVDF membrane. FIG. 5 shows the overlay of the fluorescence images of the Calcein labeled cells prior to incubation and the PE fluorescence images of the IgG-PE labeled spots on the PVDF membrane. FIG. 5A shows the PE fluorescence image of the whole PVDF membrane of the whole microwell chip area. It shows a large variation of PE fluorescence amongst the individual dots representing different amounts of the VU1D9 antibody produced by the different cells. FIG. 5B shows a larger magnification of one position, here one can clearly discern the presence of living cells (white) in the majority of the wells and a large variation of antibody production (gray) between the cells (some with no detectable production). The wells containing the cells of interest can now be located and isolated using the principle illustrated in FIG. 1. Production as a function of time can be obtained by placing a new PVDF membrane at different time points during the incubation at the bottom of the microwell chip and analyzing the printed spots. In this case recombinant EpCAM was used a capturing molecule, other capturing molecules, e.g. protein A, can be used as well. Alternatively, the produced antibodies can be captured by the bare activated PVDF membrane as well. Another possibility is to place the microwell chip subsequently on PVDF membranes that are labeled with different capturing antibodies or have different ligands on the membrane below each spot.

SPRi is used to monitor, track and quantify the secretion of antibodies label free and in real time from each of the individual cells. Selection of cells from a pool of thousands of cells can be carried out after an overnight incubation in hours instead of weeks. Screening can be performed not only on maximum secretion of product but also on intrinsic antibody parameters as affinity (K_{D}), on- and off rates (kₐ and k_{d}) respectively. The principle of using microwells in combination with SPRi as read out is illustrated in FIG. 6. The microwells with single cells are attached to a SPR sensor surface and immersed in cell culture medium. The products of the cells diffuse via the pores in the 1 μm membrane bottom of the microwell, to the SPRi sensor surface. The distance between the microwell bottom and the SPRi surface is as small as possible. To minimize the distance between the bottom of the microwells and the SPRi capturing surface a small pressure is applied to the microwell chip. The insert of the figure magnifies the area with a cell in the bottom of a well and described in more detail elsewhere (Abali et al. Anal Biochem. 2017, 531:45-47.)

To demonstrated VU1D9 antibody production by individual VU1D9 hybridoma cells, a cell suspension containing VU1D9 producing hybridoma cells were passed through the microwell chip. After this seeding process, the microwell device is connected to a SPR sensor. The SPR sensor and microwell, filled with single cells, are incubated for a certain period of time (e.g. overnight) allowing the cells to secrete specific molecules, which will be captured by ligands immobilized on the sensor surface. The incubation can be performed in an incubator or can be carried out inside the SPR imager instrument. The latter allows measurement of the secretion and production levels of the single cells in the microwells in real-time, the incubation can be carried out inside the SPRi instrument. The SPRi instrument monitors the secretion levels in real-time and label free and no additional labels are required. After completion of the incubation an SPR image of the microwell is obtained by pushing the microwells inside the evanescent field of the light source used to scan the SPRi chip as illustrated in FIGS. 3 C and D. This will generate an image of the silicon membrane of the microwell chip, which allows for the correlation of the printed spots with the microwell number and cell belonging to that spot. After completion of the incubation phase, the microwell chip is removed from the SPRi sensor for exposing the printed molecules on the sensor surface to a specific biomolecular interaction by injecting a specific analyte (e.g. the antigen) for the captured molecules that are produced by the cells.

FIG. 7 shows a SPR image of VU1D9 antibodies secreted in the microwells and printed on the sensor surface. The amount of specific product and the affinity criteria such as on- and off rate for each arrayed cell can be determined. Based on the measured SPR sensor graphs for each individual printed spot the cells of interest are selected. The microcell number and cell that belongs to the specific printed spot is determined from FIG. 3 C, D followed by isolation of the cells as depicted in FIG. 1D.

To be able to use SPR imaging in combination with the microwells the interface between the bottom of the microwell chip and the surface on which the secreted molecules are captured and analysed is of utmost importance. A constant space between both surfaces and an even pressure between both surfaces is important to accommodate even passage of the secreted molecules across all wells. Optimal configuration of this interface may differ between different materials of the capture surface and modification of these surfaces with, for example chemicals that can improve the wetting properties, can alter the specifications for this interface.

### Examples of Devices to Couple Microwells to Capture Surfaces.

FIG. 8 shows a coupling device that contains a microwell chip contained within the bottom of the cup (Panel A) and a cap (Panel B) that can be used to close the cup thereby maintain sterility and avoiding evaporation of the medium. A cell suspension can be placed onto the cup and the cells are seeded in the microwells. After cell seeding, the cap is placed on the cup and the coupling device is placed on the capture surface. FIGS. 8 C, D and E shows the placement of the coupling device on a Surface Plasmon Resonance imaging sensor. FIG. 9 shows the schematic representation of the coupling device placed on a SPRi sensor. The coupling device as described herein consists of a cup (17) that contains the microwell chip (12) and a cap (19) that closes the cup and maintains sterility and avoids evaporation of the medium. Furthermore it will need to restrict the fluidic flow from the microwells to the surface of the SPR capture surface. It is important that the microwells are mounted in the cup with the ability to move up and down to be able to position itself flat on the SPR capture surface, independently from the rest of the cup, by pushing it onto the SPRi surface. This is to ensure that a proper contact between the microwells and the capture surface that is equal across the whole microwell chip. The O-rings in the bottom of the cup (15) creates a seal on the SPR sensor surface to prevent fluid transport from the microwells to the open air. Depending on the type of SPR sensor, the O-ring can also be positioned at the top of the cup (14) where it creates a seal with the surface of the SPR holder. In this example, a flexible tape (16) preferably Peek tape, is used to connect the microwell chip with the cup. The cap is used to close the cup and at the same time it presses the microwell chip onto the sensor surface and pushes the O-rings (12, 14) in the bottom of the cup onto the SPRi surface or SPR holder. Controlled pressing and releasing the device is important to allow the secreted molecules to diffuse to the capture surface. FIG. 9B shows the cap (19) and a head (18) that creates the force to press the microwell chip onto the SPR surface (10). The cap presses the edges of the tape (16) and microwell chip (12) hereby pressing the microwell chip bottom on the sensor surface. The cup will be closed by an O-ring (13) while pressing the cap.

An example of a coupling device in which the secreted molecules are captured on a PVDF membrane is illustrated in FIG. 10. A microwell chip (1) is mounted in a plastic holder (21). After filling of the microwells with single cells, the microwell chip is placed onto the activated PVDF membrane. For this, the PVDF membrane (3) is placed on a rubber slab (20) and on top of this slab the microwells are placed such that the PVDF membrane fits exactly on the microwell bottom. In this case the PVDF membrane is 10×10 mm, which is the same size as the microwell chip. The stack is placed on a clamping unit (22). By closing the unit with the handle (23), the microwells are pushed down onto the PVDF membrane to ensure even contact between microwells and membrane across the whole chip as well as generating sufficient force on the membrane to obtain an indentation pattern of the microwell chip on the PVDF membrane. The latter is to correlate the location and microwell number with the printed molecules on the PVDF membrane.

### Simultaneous Analysis of Multiple Secreted Molecules by Single Cells.

Printing biomolecules on a sensor surface can be carried out using the technology as described in US20150306560 (Bat E, Jonkheijm P, Huskens J, Stamp for making a microarray of biomolecules). A microchip with an array of square holes is applied and filled with a hydrogel. The device will be embedded in the coupling device as described in this application. When a capture surface is connected to the coupling device with hydrogel filled microchip and filled with ligands (e.g. antibody, protein A/G etc.). FIG. 11 present 3D impressions of the microwells connected to a capture surface that contains in this case 4 different capture molecules (A, B, C and D). The ligands are stamped in an array on the capture surface. Special surface chemistry can be applied including linking via photo cross-linking. A second stamp can be made with an equally aligned chip on the sensor filled with ligand B. A third and fourth stamp will follow to connect in the corners ligands A, B, C, D, See FIG. 11. The pore of the microwell will be in close contact with the corners of the capture area A, B, C and D.

Thus, it should be understood that although the present invention has been specifically disclosed by preferred embodiments and other features, modification and variation of the invention embodied therein herein disclosed may be used by those skilled in the art, and that such modification and variations are considered to be within the scope of this invention.

1- A molecule printing device comprising:
a. a microwell plate having individual microwells each with a bottom plate wherein at least one bottom plate has at least one precisely etched pore to pass a sample liquid containing molecules from a supply side to a discharge side; and
b. a capturing surface which is connected to the microwell plate such that molecules present in the sample liquid will move from the supply side of the pore towards the discharge side and captured on the capturing surface.

2- The device according to claim 1, wherein the sample liquid contains components of interest with a slightly larger diameter than the pore such that when the sample fluid is applied to the microwell the object of interest will occlude the pore.

3- The device according to claim 2, wherein the components of interest is a cell capable of partly or completely blocking the pore.

4- The device according to claim 2, further having a means for retrieving captured components of interest.

5- The device according to claim 2, wherein the molecules are secreted by the components of interest.

6- The device of claim 4 wherein the retrieving means is by a punching means of the bottom plate or a cell picking means using a micropipette.

7- The device of claim 1 having a capturing surface composition of any known surface for capturing molecules

8- The device of claim 7 having a capturing surface composition selected from the group consisting of glass, metal, ceramic, non-ceramic, organic, non-organic, nitrocellulose, paper, PVDF and combinations thereof.

9- The device of claim 7, wherein the capturing surface contains capturing molecules that are able to capture the molecules present in the liquid.

10- The device of claim 9 having capturing molecules selected from the group consisting of antibodies, antigens, DNA, RNA, biotin, streptavidin and combinations thereof.

11- The device of claim 9 wherein the capturing surface is covered with a pattern of capturing molecules for a single microwell.

12- The device of claim 7 wherein the location of the microwells can be correlated to the printed molecules on the surface.

13- The device of claim 5 having a coupling means for maintaining a sterile and pressurized enclosure on the capturing surface while monitoring secreted molecules by surface plasmon resonance imaging (SPRi) comprising:
a. a cup containing a microwell plate; and
b. a cap to maintain a pressurized and sterile enclosure.

14- The device of claim 13 wherein the pressure in the enclosure is determined by a thickness in the cap.

15- The device of claim 13 where the cup is bound to the microwell using tape.

16- The device of claim 15 wherein the tape is PEEK.

17- The device of claim 13 further having a means for connecting the microwell to the capture surface during transfer of molecules from the microwell to the capturing surface as a printed molecule, said means characterized by controlling a distance between the microwell and the capture surface.

18- The device of claim 17 wherein the distance is controlled by applying pressure to the top of the cap.
